(19) United States
(12) Patent Application Publication
(10) Pub. No.: US 2020/0329952 A1
(43) Pub. Date: Oct. 22, 2020

(54) ENDOSCOPE HAVING A PIVOTABLE HANDLE PART

(71) Applicant: OLYMPUS WINTER & IBE GMBH, Hamburg (DE)

(72) Inventor: Gregor KIEDROWSKI, Hamburg (DE)

(73) Assignee: OLYMPUS WINTER & IBE GMBH, Hamburg (DE)

(21) Appl. No.: 16/763,265

(22) PCT Filed: Dec. 7, 2018
(86) PCT No.: PCT/EP2018/084020

§ 371 Date: May 12, 2020

(30) Foreign Application Priority Data
Dec. 7, 2017 (DE) 10 2017 129 188.1

(51) Int. Cl. A61B 1/00; A61B 1/07; A61B 1/01

(52) U.S. Cl. CPC A61B 1/00066; A61B 1/01; A61B 1/07; A61B 1/00126

(57) ABSTRACT

A medical endoscope including: an elongate shaft; and a main body arranged proximal to the shaft, wherein an optical waveguide extends through the shaft in the longitudinal direction, the optical wave guide being bent in the main body into a connecting piece extending from the main body, the main body comprises at least one handle arranged adjacent to the connecting piece in a longitudinal direction of the main body, and the handle being rotatable into a plurality of rotational positions around a longitudinal axis of the shaft.

### CROSS-REFERENCE TO RELATED APPLICATION

The present application is based upon and claims the benefit of priority from PCT/EP2018/084020 filed on Dec. 7, 2018, which claims benefit to DE 10 2017 129 188.1 filed on Dec. 7, 2017, the entire contents of each of which are incorporated herein by reference.

### BACKGROUND

### Field

The present application relates generally to a medical endoscope and more specifically to an endoscope with a shaft part and a main body arranged proximal to the shaft part, wherein an optical waveguide extends through the shaft part in the longitudinal direction, which optical waveguide is guided at an angle in the main body into a connecting piece, and the main body comprises at least one handle part, which is arranged adjacent to the connecting piece in the longitudinal direction of the endoscope.

### Prior Art

Medical endoscopes are instruments with an elongated, tubular, flexible or rigid shaft, and optics, which are used for the optical, minimally invasive examination of internal spaces of the human body. Endoscopes are used, e.g., in urology and are designated there as ureteroscopes. The shaft tube of a ureteroscope should be stiff to enable the direction of the distal end from the proximal end. The shaft tube is therefore generally rigid and has an outer metal tube, which provides the necessary stiffness and surrounds the inwardly-lying parts of the shaft part in a liquid-tight manner.

Generally, at least one viewing device is provided in the interior of the shaft tube, and optical waveguides for lighting, and can also include a working channel, which enables the insertion of instruments, like catheters, forceps, stone catchers, lithotriptors (stone fragmentation devices) etc. At least one fiber optic bundle can be provided in addition to illuminate the working area lying distally in front of the endoscope. An example of such an endoscope is disclosed in DE 43 35 783 A1.

The optical wave guide is generally guided in the proximal end area of endoscopes in a connecting piece or optical waveguide connector, branching off of the shaft or from the main body of the endoscope, out of the interior of the endoscope. An optical fiber connection cable, which leads, e.g., to a cold light source, can be connected to the connecting piece.

To enable comfortable use of the endoscope by skilled medical personnel, many endoscopes, such as endoscopes with a compact design, have two ergonomically-shaped handle parts, made from plastic material, in their proximal end area. In conventional endoscopes (e.g., ureteroscopes), input sockets for the working channel(s) and the connecting piece for the optical waveguide can be arranged between the two handle parts. In compactly designed endoscopes, the connecting piece can thereby be arranged so closely adjacent and parallel to the distal handle part that connecting the fiber optic connection cable can be uncomfortable. While this design contributes to the compactness and ease of use of the endoscope when operated during an examination, and can also shield the user from heat potentially generated at the optical waveguide, the design is rather annoying during the assembly of the instrument under certain circumstances.

### SUMMARY

An object is thus to provide a medical endoscope with a compact design, in which the space-saving adjacent arrangement of optical waveguide connecting piece and the distal handle part can be retained, and the connection of the optical waveguide during assembly of the instrument is facilitated.

Such problem can be solved by a medical endoscope with an elongated shaft part and a main body arranged proximal to the shaft part, wherein an optical waveguide extends through the shaft part in the longitudinal direction, which optical waveguide is bent, in the main body into a connecting piece, and the main body comprises at least one handle part, which is arranged adjacent to the connecting piece in the longitudinal direction of the endoscope, wherein the handle part can be brought into a plurality of rotational positions around the longitudinal axis of the endoscope.

Since the handle part, arranged adjacent to the optical waveguide connecting piece, is rotatable about the longitudinal axis of the endoscope, the handle part can be moved so far away from the outer end of the connecting piece during the attachment of the fiber optic cable that a comfortable plugging in or screwing on of the cable is possible. At the same time, the compact design of the endoscope is not compromised. During use of the endoscope, the skilled medical personnel can therefore continue to comfortably reach all handle parts and operating elements which are arranged on the main body.

The medical endoscope can be a gynecological or urological endoscope. The endoscope can be, e.g., a ureteroscope, arthroscope or cystoscope. Alternatively, the endoscope can also be a gastroscope, colonoscope or bronchoscope. The endoscope can be configured as a resectoscope. In an embodiment, the medical endoscope is a ureteroscope.

In typical endoscopic design, the endoscope has an elongated shaft part. The shaft part or tubular shaft of the endoscope can have, e.g., a length of at least approximately 150 mm. e.g., a length between 150 mm and 400 mm, such as between 150 mm and 250 mm. The shaft diameter can be, e.g., approximately 4 to 10 mm. A very long and thin configuration of the tubular shaft can result from such configuration.

Endoscopes can be configured to be both rigid and also flexible. Thus, ureteroscopes, arthroscopes, and cystoscopes can be configured as rigid, while gastroscopes, colonoscopes, or bronchoscopes can be configured as flexible. The shaft part of the endoscope can thus be rigid, flexible, or semi-flexible, i.e., merely flexible in sections. The shaft part can be a rigid shaft part. The shaft part can be configured as tubular. The shaft part can be made from metal.

An optical waveguide can extend through the shaft part in the longitudinal direction. The optical waveguide generally extends across the entire length of the shaft part, i.e., from its proximal to its distal end. The optical waveguide is suited for guiding light from a light source to the distal end of the shaft part. Suitable optical waveguides, having a plurality of individual fibers, are known to those having ordinary skill in the art and are routinely used in endoscopy or ureteroscopy. The at least one optical waveguide can be configured as an optical fiber bundle, i.e., as a bundle made of optical fibers. An optical fiber bundle has in each case a plurality of optical fibers, e.g., a plurality of glass fibers. Optical fibers can be, e.g., glass fibers or fibers based on plastic material. The optical waveguide or the optical fiber bundle can be coupled to an external light source.

The cross-sectional shape of the optical waveguide can vary. Thus, the optical waveguide can have a round cross section, and/or extend in an optical waveguide channel within the shaft part. However, the optical waveguide can also adapt to the free space in the shaft part and fill in the same.

The endoscope additionally comprises a main body (also called the operating part) arranged proximal to the shaft part. This main body comprises at least one handle part, a connecting piece for the optical waveguide, and optionally one or more input sockets for working channels. The optical waveguide, optics, and the working channels also extend through the main body at least in sections. The main body can have a larger diameter than the shaft part.

The optical waveguide is angled in the main body (out of the longitudinal axis of the endoscope) into a connecting piece. In general, the connecting piece (also optical fiber connection socket) is arranged on the main body in such a way that the longitudinal axis of the connecting piece extends transverse to the longitudinal axis of the endoscope. The angle between the longitudinal axis of the endoscope and the longitudinal axis of the connecting piece is generally approximately 90°. Alternatively, however, the angle can also be smaller, e.g., between 30° and 90°, starting from the proximal leg of the longitudinal axis of the endoscope. The connecting piece can generally have a part of a plug or screw connection. A fiber optic cable with a complementary part can be plugged in or screwed on to the same.

In addition, the main body comprises at least one handle part. In an embodiment, the endoscope can have more than one handle part. e.g., two handle parts. The handle part(s) can be arranged so that both can be grasped with the same hand. Thus, one handle part, e.g., arranged on the main body, can be configured in such a way that it can be held using the index finger, middle finger, ring finger, and/or little finger, and/or another handle part can be arranged on the ocular, which can be held using the thumb. The handle pats can have a corresponding ergonomic shape, e.g., can be adapted in shape to the finger used for operating the same.

The handle part arranged on the main body can be arranged adjacent to the connecting piece in the longitudinal direction of the endoscope. Stated another way, the handle part and the connecting piece can be adjacent on the main body. When viewed from the distal or proximal end, the handle part and connecting piece can be arranged one behind the other. The longitudinal axis of the handle part can generally be parallel to the longitudinal axis of the connecting piece in an active position described herein. However, to adjust the ergonomics of the handle part to the handle system of the endoscope, there can also be deviations from this parallelism. Thus, the longitudinal axis of the handle part can correspond. e.g., to the longitudinal axis of the connecting piece, which can be displaced in parallel and tilted by 0° to 35°. The active position of the handle part is the position, in which the endoscope can be used for surgical procedures. The handle part in the active position can be arranged so closely adjacent to the connecting piece, that the endoscope can have a particularly compact design. At the same time, however, the compact arrangement in some embodiments leads to the fact that the fiber optic cable can only be fixed to the connecting piece with small movements in the active position. The distance between the connecting piece can be, e.g., 0.2 cm to 3 cm, such as 0.5 cm to 2 cm, e.g., approximately 1.5 cm.

The rotatably handle part described herein is the handle component, which can extend in only one direction out the longitudinal axis of the endoscope, such as in the direction which, as described above, is substantially parallel to the connecting piece in an active position.

The rotatable handle part can be part of a larger handle, which extends. e.g., on the opposite side of the main body in the opposite direction.

In order to guarantee the comfortable accessibility of the connecting piece despite the compact design of the endoscope, the handle part is rotatable about the longitudinal axis of the endoscope. By rotating the handle part out of an active position, the outer end of the connecting piece is freely accessible, as the handle part is no longer located directly adjacent to the outer end of the connecting piece after the rotation is carried out. The spacing between the outer end of the connecting piece and the (outer end of the) handle part can be enlarged by the rotation by, e.g., at least 1 cm, such as at least 2 cm or at least 3 cm. The “outer end” is herein in each case the end of a radial component which is spaced the farthest apart from a midpoint, i.e., from the shaft-like element of the main body, such as its longitudinal axis.

The handle part can thus be brought into a plurality of rotational positions about the longitudinal axis of the endoscope. The handle part can be brought into a plurality of rotational positions relative to the connecting piece likewise arranged on the main body. The handle part can be steplessly rotatable (within a certain angular range) about the longitudinal axis of the endoscope. The handle part can be rotatable, e.g., in an angular range from 0° to 90°, such as from 0° to 45° about the longitudinal axis, wherein 0° is formed by the active position.

The handle or the handle part can have a rotational element, which is mounted to be rotatable about the longitudinal axis of the endoscope and arranged on the main body or a part of the same. Mediated by the rotatability of the rotational element, the handle or the handle part can thus be rotated about the longitudinal axis of the main body or brought into a plurality of rotational positions. The rotational element can surround a shaft-like element of the main body for this purpose. The rotational element can be, e.g., ring shaped. By rotating the, e.g., ring-shaped rotational element about the shaft-like element of the main body, the handle part can be brought into a plurality of rotational positions. The handle part or handle and the ring-shaped rotational element can be configured as one piece. Alternatively, the handle part or the handle can be applied to the ring-shaped rotational element.

The rotational element or a section of the rotational element can be arranged in a groove encircling the main body or a part of the main body. The rotational element can be displaced in the groove in the circumferential direction of the main body. The displacement in the circumferential direction causes the rotation of the handle part described herein. The groove can have one or more rotation limiting elements, which can be complementary to corresponding rotation limiting elements arranged on the rotational element. With the aid of the rotation limiting elements in the groove and on the rotational element, the rotational angle of the handle part or of the handle can be limited about the longitudinal axis of the endoscope.

By rotating the handle part, the handle part is brought into an active position, in which the instrument can be used for operations. In the active position, the handle part or its longitudinal axis can be, as described above, substantially parallel to the connecting piece or its longitudinal axis. By this means, the instrument can be comfortably and securely held by skilled medical personnel on the one hand, on the other hand, the connecting piece is also less accessible in this position. The latter also represents a protection for this connection against damages or loosening between the optical cable and the instrument.

The longitudinal axis of the handle part can lie, in the active position described herein, in a plane (“active plane”) with the longitudinal axis of the connecting piece and the longitudinal axis of the shaft part. Thus, the handle part or its longitudinal axis can be brought into a plurality of rotational positions relative to the plane. Since these rotational positions move the handle part out of the normal handle position used during operation, these rotational positions are no longer designated as an active position. In these transitional rotational positions, the handle part is moved so far away from the outer end of the connecting piece, that this end is freely accessible and an optical cable can also be applied (connected) to the end using larger rotational movements. The handle part can thus be brought into a connection position, in which the longitudinal axis of the handle part forms an angle of 30° to 45°, such as 45°, with the plane described herein. It has been demonstrated that a comfortable application of the optical cable is possible if the handle part has assumed an angle of 30° to the active plane.

The handle part can be automatically returned into the active position if the handle part is not held (retained) in the connection position or in a transitional rotational position by a counter pressure. The handle part can be held in the active position, e. g., by the reset force of a spring element. Simultaneously or alternatively, the handle part can be brought out of the connection position into the active position by the reset force of a spring element. These two functions can be provided by the same spring element(s) or by different spring elements.

One spring element, which is suited for bringing the handle part from the connection position or the transitional rotational position into the active position, can be a spring element whose reset force acts in the circumferential direction of the main body in the direction of the position of the active position. Such a spring element can also be suited for holding the handle part in the active position and for preventing the handle part from moving out of the active position into a transitional rotational position or into the connection position without the effect of an external force that exceeds gravity.

Another spring element, which is suited for holding the handle part in the active position, can be a spring element whose reset force acts radially in the main body. The spring element can exert a force on a complementary element to lock the handle part in the active position. The spring element can thereby be arranged on the handle part, e.g., on the rotational element of the handle part, and the complementary element can be arranged on another part of the main body, or vice versa. The spring element can be, e.g., a ball-spring element, the complementary element can be a depression at least partially complementary in shape to the ball.

The endoscope can additionally comprise a locking element or latching element, which prevents an unintentional rotation of the handle part out of the active position. The locking element can be a spring element described at another point, which is configured to hold the handle part in the active position. Alternatively, however, the locking element can also be formed by another retaining element. Thus, the handle part can be held, e.g., by a pin-shaped or otherwise shaped element, e.g., a guide pin, in the active position, which can be arranged on the main body and engages in a depression on the rotational element or vice versa.

In one embodiment, the main body can comprise a guide pin, which engages in the active position with a depression, which is arranged on the rotational element. The guide pin can extend outwardly in the radial direction from the shaft-like element of the main body. The outer end area of the guide bolt can be arranged in a groove. i.e., can engage with the groove, which is formed in a groove element. The groove element can be part of the rotational element. In this way, the groove element can be connected immovably to the handle part. A rotation of the handle part about the longitudinal axis of the main body correspondingly causes a rotation of the groove element comprised by the rotational element. By this means, a displacement is caused of the guide bolt in the groove. It is likewise conceivable that the guide pin can be connected immovably to the handle part and the rotational element, while the groove is arranged on a non-rotatable section of the main body. Thus, in one embodiment, either the groove element or the guide pin can be connected immovably to the handle part.

The grove can extend in the circumferential direction of the main body or the shaft-like element of the main body. In order to limit the rotational angular range of the handle part, the groove does not have to extend around the entire circumference of the main body or the shaft-like element of the main body (i.e., not in an angular range of 360), but instead can span an angular range of approximately 180° or less, such as approximately 90 or approximately 45 or approximately 30. In this case, 0° is the respective active position.

The groove can have the above-mentioned depression, which acts as a locking element. The depression can be complementary in shape, at least in sections, to the guide pin. The depression can be, e.g., a partially circular shaped depression.

Pressure can be applied to the guide pin so that it can move decentrally in the groove and/or on one side, from the distal or proximal side (in the direction of the side of the groove, in which the depression is arranged). In this way, the guide pin can engage with the depression as soon as the guide pin is arranged in the groove due to the rotation to the rotational position, in which the depression and the guide pin are arranged axially apart from one another.

The “rotatable” handle part, which can be brought into a plurality of rotational positions, can be part of a larger handle. The longitudinal axis of the endoscope can cross the handle in its center third, and can extend transverse to the longitudinal axis of the handle part. In other words, the handle can have, on the side of the longitudinal axis of the endoscope opposite the “rotatable” handle part, another handle part (an extension of the handle part). In this way, the endoscope can be held more securely in the hand at this point during use.

Also provided is a medical examination method, in which the endoscopes described above are used. Such method includes the preparation of the instrument by connecting an optical cable, wherein the handle part of the endoscope is brought out of a plane, which is formed by the longitudinal axis of the connecting piece, the longitudinal axis of the shaft part, and the longitudinal axis of the handle part in an active position, into a connection position, in which the longitudinal axis of the handle part forms an angle to the plane, such as an angle of 30° to 45°, such as 45°. The handle part is subsequently brought back into the active position in preparation for using the instrument in an operation. The instrument can then be used for a minimally invasive examination.

Also provided is a medical treatment method in which the endoscopes described above are used.

### BRIEF DESCRIPTION OF THE FIGURES

Embodiments are schematically depicted in the drawings, where:

FIG. 1 illustrates a schematic side view of an endoscope with a distal handle part and another proximal handle part, which is arranged on the ocular;

FIG. 2 illustrates a simplified, schematic sectional depiction of the endoscope from FIG. 1 transverse to the longitudinal axis of the endoscope, in which the distal handle part is clear and in an active position;

FIG. 3 illustrates a simplified, schematic sectional depiction of the endoscope from FIG. 1 transverse to the longitudinal axis of the endoscope, in which the distal handle part is clear and in a transitional rotational position;

FIG. 4 illustrates a simplified, schematic sectional depiction of the endoscope from FIG. 1 transverse to the longitudinal axis of the endoscope, in which the distal handle part is clear and in a connection position;

FIG. 5 illustrates a sectional side view of the main body of the endoscope from FIG. 1, in which the handle part and connecting piece are clear,

FIG. 6 illustrates a perspective, sectional view of the main body of the endoscope from FIG. 1, in which the handle part and connecting piece are clear; and

FIG. 7 illustrates an outer view of the main body of the endoscope from FIG. 1, wherein the outer cap of the main body is removed in order to make the arrangement of a guide pin in a groove visible.

### DETAILED DESCRIPTION OF THE INVENTION

FIG. 1 shows a schematic side view of an endoscope 10 with a distal handle part 20 and a proximal handle part 22, which is arranged on the ocular 44 with the proximal handle part 22. Endoscope 10 has a longitudinally extended shaft part 12. Main body 13 and the ocular 44 are arranged proximal to shaft part 12.

Main body 13 comprises a shaft-like element 34, on which connecting piece 18 and an input socket 36 are arranged. Connecting piece 18 is provided for connecting a cold optical cable. By means of the optical cable and the optical waveguide (not shown here) extending in connecting piece 18 and shaft part 12, the operational area can be illuminated during an operation. The connection between connecting piece 18 and an optical cable can be configured as a screw connection. It is clear that the spacing between connecting piece 18 and handle part 20 is very small in this arrangement. Thus, the connection between connecting piece 18 and the optical cable (not depicted here) is not protected on the one hand, and the attachment of the cable at connecting piece 18 is impeded on the other hand.

Input socket 36 comprises a working channel (not depicted here). The working channel extends through shaft part 12 and is bent into input socket 36. Different through passage instruments, which are used in an examination, can be inserted into the instrument through input socket 36.

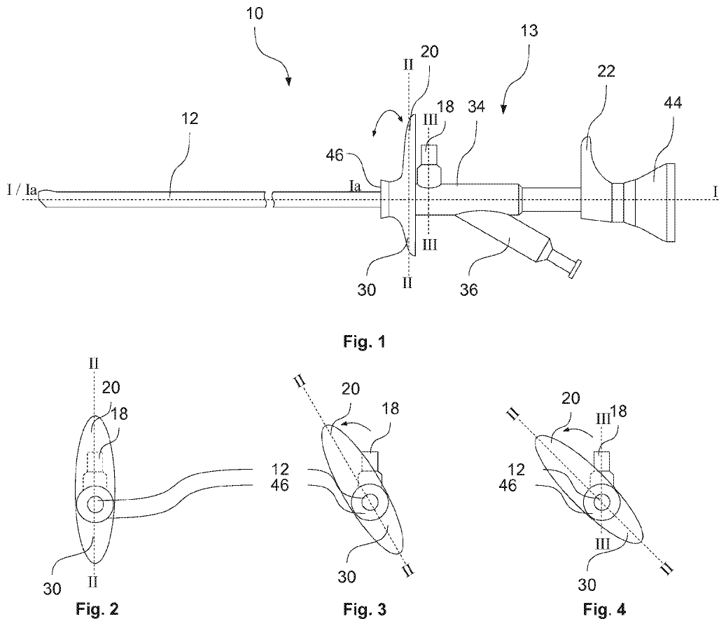

Main body 13 additionally has a handle part 20, which is arranged parallel to connecting piece 18 on shaft-like element 34 or in main body 13. Handle part 20 is part of a handle, which has an extension 30 of the handle part on the side of the main body opposite the handle part. Due to this shape of the handle, a particularly secure and comfortable handling is guaranteed. Handle part 20 and its extension 30 are mutually configured as a one-piece plastic polymer part. Handle part 20 or the entire handle can be brought into a plurality of rotational positions about longitudinal axis I-I of endoscope 10. Handle part 20 is thereby in the active position in the position shown. In the active position, longitudinal axis II-II of the handle part is arranged in a mutual plane with longitudinal axis III-III of the connecting piece and longitudinal axis Ia-Ia of the shaft part, which is part of longitudinal axis I-I of the endoscope. As is clear from the following FIGS. 2 to 4, handle part 20 can be brought into a plurality of rotational positions about longitudinal axis I-I of endoscope 10 relative to connecting piece 18 and relative to the plane (“active plane”) described above.

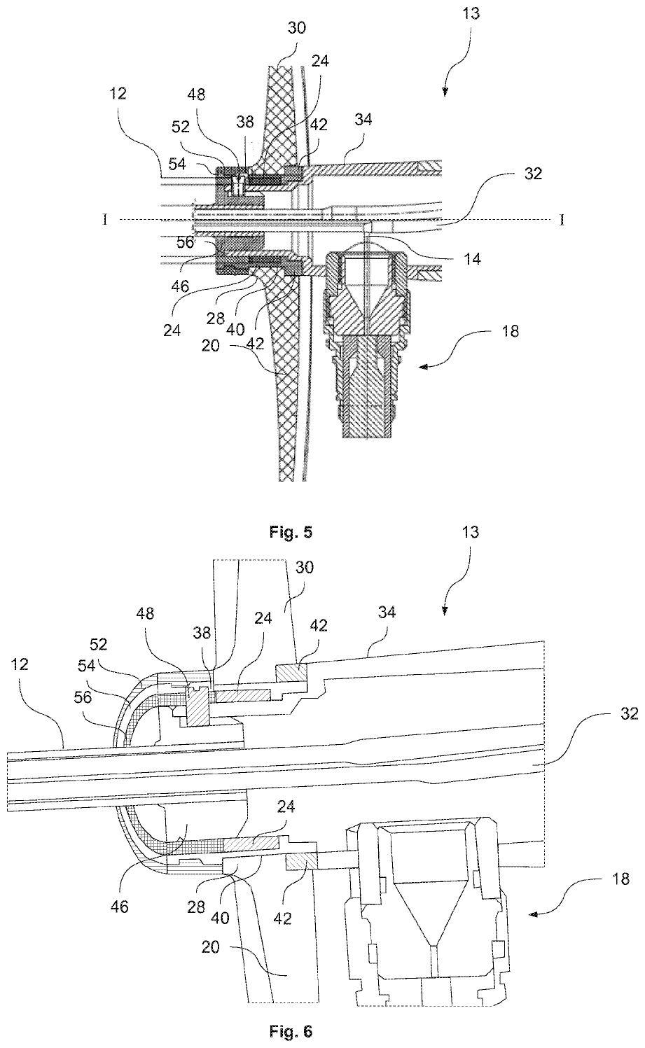

Main body 13 additionally comprises a sealing element 46. The sealing element is configured as a sealing ring and is depicted in greater detail in FIGS. 5 and 6. Sealing element 46 is arranged in the distal end area of main body 13 and seals the interior of main body 13 against contamination. In addition, it is also conceivable that sealing element 46 can function as a fixing means for fixing shaft part 12 to main body 13 or to certain components of main body 13.

FIGS. 2, 3, and 4 show sectional depictions of endoscope 10 from FIG. 1 transverse to longitudinal axis I-I of endoscope 10 shown in FIG. 1. In each case, a strongly schematic cross section of shaft 12, sealing element 46 arranged proximally thereupon, and distal handle part 20 are clear. Working channels and other detail elements, like optical waveguides extending in shaft part 12, are not depicted. Handle part 20 and its extension 30 can be brought into a plurality of rotational positions about longitudinal axis I-I of endoscope 10 shown in FIG. 1, of which three exemplary rotational positions are depicted in FIGS. 2, 3, and 4. The more handle part 20 is rotated out of the active position, the better the accessibility to connecting piece 18. Ocular 44 and proximal handle part 22 are not depicted in these illustrations.

FIG. 2 shows a rotational position in which distal handle part 20 is arranged in the active position. Connecting piece 18 is indicated, which is not visible from this perspective behind handle part 20 and is difficult to access in the active position. In this active position, handle part 20 is arranged on the main body parallel to connecting part 18. Longitudinal axis II-II of handle par 20 forms a plane with longitudinal axes I-I and III-III (not depicted) of endoscope 10 and connecting piece 18.

FIG. 3 shows a rotational position in which handle part 20 is arranged in a transitional rotational position or a connection position. Longitudinal axis II-II of handle part 20 forms an angle of approximately 30° to the plane defined above. The outer end of connection piece 18 is completely visible and easily accessible.

FIG. 4 shows a rotational position, in which handle part 20 is arranged in a connection position. Longitudinal axis II-II of handle part 20 forms an angle of approximately 45° to the plane defined above. The outer end and other areas of connecting piece 18 are completely visible and particularly easy to access.

FIG. 5 shows a sectional side view of main body 13 of endoscope 10 from FIG. 1, in which handle part 20 and connecting piece 18 are clear. The proximal end of shaft part 12 is also shown in sections.

Main body 13 has the connecting piece 18, through which an optical waveguide 14 passes. Optical waveguide 14 extends from the outer end of connecting piece 18 into the interior of shaft-like element 34 of main body 13 and angles there into shaft part 12 of endoscope 10. A working channel 32 additionally extends through shaft-like element 34 and shaft part 12.

Main body 13 additionally has the handle part 20, which forms a handle with an extension 30 on the opposite side of longitudinal axis I-I of endoscope 10, with the aid of which endoscope 10 can be securely and comfortably held. The handle has a ring-shaped rotational element 28. Rotational element 28 forms the center part of the handle, which lies between handle part 20 and its extension 30. Rotational element 28 surrounds shaft-like element 34 of main body 13. Sections of rotational element 28 thereby run in a groove 40. Rotational element 28 is rotatably mounted in groove 40. Additional rotation limiting elements 42, which limit the free rotatability of handle part 20 to an angular range of 0° to 45°, are arranged in groove 40.

The sealing element 46, which seals the interior of main body 13, is arranged at the distal end of main body 13. Sealing element 46 is configured as a sealing ring.

Furthermore, a guide pin 48 is mounted on an area of sealing element 46 arranged in the interior of shaft-like element 34. The longitudinal axis of guide pin 48 extends in the radial direction. The outer end or the outer end area of the guide pin engages with a groove 38 for the guide pin, i.e., the outer end or the outer end area of guide pin 48 runs in a groove 38 for the guide pin. The outer end or the outer end area is the end or the end area of the guide pin facing away from the longitudinal axis of the endoscope. Groove 38 is arranged in a groove element 54, which surrounds sealing element 46. Groove 38 extends in the circumferential direction of main body 13, such as in the circumferential direction at a substantially constant radius. Groove 38 thereby has a width which corresponds at least to the width of guide pin 48. The width of groove 38 is the extension of the groove in the longitudinal direction of the main body or of the medical endoscope. Groove 38 has a depth which ensures that a stable engagement exists between guide pin 48 and groove 38, i.e., that guide pin 48 cannot slip out of groove 38. The depth of groove 38 is the extension of the groove in the radial direction. Groove 38 has a length (in the circumferential direction of main body 13), which enables a free rotatability of handle part 20 in a desired angular range, e.g., an angular range from 0° to 45°. The ends of groove 38 can act as rotation limiting elements in the circumferential direction. Thus, groove 38 can span an angular range from 0° to 45° of the main body.

FIG. 6 shows a perspective sectional view of medical endoscope 10 also depicted in FIG. 5. It is clear, that sealing element 46 extends in a ring shape about the extension of shaft part 12. In other words, sealing element 46 is configured as a sealing ring. For reasons of simplification, optical waveguide 14, extending through shaft part 12, main body 13, and connecting piece 18, is not depicted in the sectional view of FIG. 6.

Furthermore, it is clear that endoscope 10 comprises two or more spring elements 24 and two or more rotation limiting elements 42. The reset force of spring elements 24 acts in the circumferential direction in the direction of the active position.

In addition, groove element 54 is clear, which is arranged as a cylinder in the distal end area of main body 13 and extends around sealing element 46. In the embodiment shown, sealing element 46 and groove element 54 are separated by a separating element 56. Groove element 54 comprises groove 38, in which guide pin 48 is arranged. Groove 38 and guide pin 48 are moveable relative to one another, in particular, guide pin 48 is moveable from one end of the groove to the other. In the present case, the movement of guide pin 48 occurs in groove 38 due to a rotation of handle part 20. The rotation of handle part 20 causes a corresponding rotation of groove element 54, as groove element 54 and handle part 20 are immovably connected to one another via ring-shaped rotational element 28.

Endoscope 10 additionally has an outer cap 52. Outer cap 52 functions for protecting the elements lying thereunder and to fix the same.

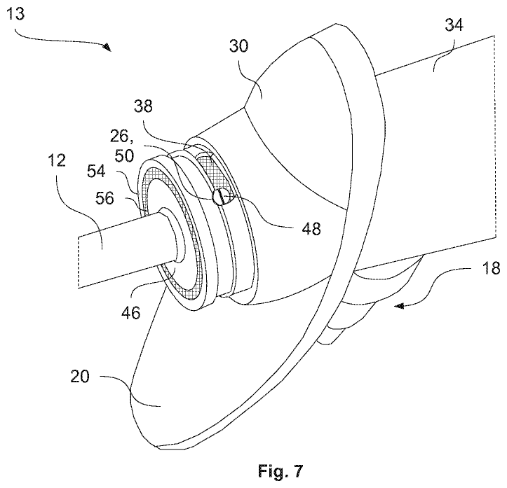

FIG. 7 shows an outer view of main body 13 of endoscope 10 from FIG. 1, wherein outer cap 56 of main body 13 is removed, in order to make the arrangement of guide pin 48 in groove 38 visible. It is clear, that groove 38 extends in the circumferential direction of main body 13. Groove 38 thereby extends across an angular range of less than 45°. However, it is also conceivable that the groove extends over an angular range longer than 45° or more.

Groove 38 has a depression 50. By snapping radial guide pin 48 into depression 50, it is ensured that handle part 20 remains in the active position during the use by skilled medical personnel, e.g., in an operation. The depression thus acts as a locking element 26. By using a manual counter pressure in the circumferential direction. e.g., on handle part 20.g uide pin 48 can be moved out of depression 50 and subsequently brought to the other end of groove 38. In the active position, it is clear that handle part 20 and connecting piece 18 are arranged closely one behind the other.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMERALS

10 Medical endoscope
12 Shaft part
13 Main body
14 Optical waveguide
16 Proximal end area
18 Connecting piece
20 Handle part
22 Proximal handle part
24 Spring element
26 Locking element
28 Ring-shaped rotational element
30 Extension of handle part
32 Working channel
34 Shaft-like element
36 Input socket of working channel
38 Groove for guide pin
40 Groove for ring-shaped rotational element
42 Rotation limiting element
44 Ocular
46 Sealing element
48 Guide pin
50 Depression
52 Outer cap
54 Groove element
56 Separating element

What is claimed is:

1. A medical endoscope comprising:
an elongate shaft; and
a main body arranged proximal to the shaft,
wherein an optical waveguide extends through the shaft in the longitudinal direction, the optical wave guide being bent in the main body into a connecting piece extending from the main body,
the main body comprises at least one handle arranged adjacent to the connecting piece in a longitudinal direction of the main body, and
the handle being rotatable into a plurality of rotational positions around a longitudinal axis of the shaft.

2. The medical endoscope according to claim 1, wherein a longitudinal axis of the handle is in a plane with a longitudinal axis of the connecting piece and the longitudinal axis of the shaft in an active position, and is rotatable into the plurality of rotational positions relative to the plane.

3. The medical endoscope according to claim 2, wherein the handle is rotatable into a connection position, in which the longitudinal axis of the handle forms an angle of 30° to 45° to the plane.

4. The medical endoscope according to claim 3, further comprising a spring having a reset force configured to one or more of hold the handle in the active position and bring the handle out of the connection position into the active position.

5. The medical endoscope according to claim 2, further comprising a locking surface configured to prevent an unintentional rotation of the handle out of the active position.

6. The medical endoscope according to claim 1, further comprising a radial guide pin, wherein an outer end area of the guide pin engages in a groove comprised by a groove element and the guide pin or the groove element is immovably connected to the handle.

7. The medical endoscope according to claim 6, wherein the groove extends in a circumferential direction of the main body.

8. The medical endoscope according to claim 6, wherein the groove has a depression which functions as a locking element.

9. The medical endoscope according to claim 1, wherein the handle has a ring-shaped rotational element arranged at least in sections in a groove surrounding a portion of the main body.

10. The medical endoscope according to claim 9, wherein the groove has one or more rotation limiting elements

11. The medical endoscope according to claim 3, wherein the handle is rotatable into the connection position, in which the longitudinal axis of the handle forms an angle of 45° to the plane.

12. The medical endoscope according to claim 8, wherein the depression (50) is preferably complementary in shape to the guide pin (48) at least in sections.
